(19) United States
(12) Patent Application Publication
(10) Pub. No.: US 2011/0313541 A1
(43) Pub. Date: Dec. 22, 2011

(54) METHODS FOR GENERATING CARTILAGE TISSUE

(76) Inventors: James E. Dennis, Cleveland Heights, OH (US); Amad A. Awadallah, Cleveland Heights, OH (US); Amold I. Caplan, Cleveland Heights, OH (US); Mark Weidenbecher, Cleveland, OH (US)

(21) Appl. No.: 12/516,859

(22) PCT Filed: Nov. 29, 2007
(86) PCT No.: PCT/US07/85892

§ 371 Date: Nov. 11, 2009

Related U.S. Application Data
(60) Provisional application No. 60/861,627, filed on Nov. 29, 2006.

(51) Int. Cl. A61F 2/02; C12N 5/077

(52) U.S. Cl. 623/23.72; 435/404; 435/405

(57) ABSTRACT

A hyaline-like, single layer cartilage tissue construct includes chondrogenic cells dispersed within an endogenously produced extracellular matrix. The single layer cartilage tissue construct has a glycosaminoglycan content substantially equal to the glycosaminoglycan content of native cartilage tissue. A method for generating a single layer cartilage tissue construct includes isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is then cultured in a serum-free culture medium for a time sufficient to permit the population of chondrogenic cells to differentiate and form the single layer cartilage tissue construct.

### RELATED APPLICATION

This application claims priority from U.S. Provisional Application No. 60/861,627, filed Nov. 29, 2006, the subject matter which is incorporated herein by reference.

### GOVERNMENT FUNDING

This invention was made with government support under Grant No. NIH DE015322-01 awarded by the National Institutes of Health. The United States government has certain rights in the invention.

### TECHNICAL FIELD

The present invention relates generally to methods and compositions for generating and repairing cartilage, and more particularly to methods and compositions for generating layered cartilage tissue constructs comprising chondrogenic cells dispersed within an endogenously produced extracellular matrix.

### BACKGROUND OF THE INVENTION

Articular cartilage has a minimal ability to heal and therefore has a tendency to accumulate damage over time. Accumulated minor damage, normal wear, and major damage often result in severely damaged cartilage that no longer provides structural support and results in significant pain and/or loss of joint movement. Interventional options for treating damaged cartilage typically include non-steroidal anti-inflammatory drugs, injection of hyaluronic acid, dietary changes, exercise, and, if the condition worsens, surgical intervention. Surgical intervention may include debridement, mosaiplasty, microfracture, and methods that use tissue engineering principles to repair damaged cartilage. If these interventions fail, the final option is often total joint arthroplasty.

When using tissue engineering methods, for example, a source of cartilage is needed. Typical sources of cartilage include mesenchymal stem cells and chondrocytes isolated from different parts of the body. Once isolated, these sources of chondrocytic cells need to be maintained and/or expanded in culture to obtain cell numbers sufficient for repair. Several methods are known that describe the use of serum-containing medium for cell expansion; however, the potential for these cells to differentiate into cartilage is unclear. Some reports indicate that chondrocytes quickly lose chondrogenic potential with passing in culture, while others describe specific culture conditions where expanded chondrocytes retain differentiation potential.

Once expanded, chondrogenic cells need to be delivered to a matrix that promotes, guides, and adheres the cells to a repair site. While the technology exists to prepare cartilage occupying a small area (e.g., approximately 1 cm in diameter), there is no described method to reproducibly produce cartilage tissue having a larger (i.e., greater than 2-5 cm) diameter and a thickness greater than 2 mm.

### SUMMARY OF THE INVENTION

According to one aspect of the present invention, a hyaline-like, single layer cartilage tissue construct comprises chondrogenic cells dispersed within an endogenously produced extracellular matrix. The single layer cartilage tissue construct has a glycosaminoglycan content substantially equal to the glycosaminoglycan content of native cartilage tissue.

According to another aspect of the present invention, a method is provided for generating a single layer cartilage tissue construct. One step of the method comprises isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is then cultured in a serum-free culture medium for a time sufficient to permit the population of chondrogenic cells to differentiate and form the single layer cartilage tissue construct.

According to another aspect of the present invention, a single layer cartilage tissue construct is produced by a method comprising the steps of isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is then cultured in a serum-free culture medium for a time sufficient to permit the population of chondrogenic cells to differentiate and form the single layer cartilage tissue construct.

According to another aspect of the present invention, a hyaline-like, multilayer cartilage tissue construct comprises a plurality of layers having a glycosaminoglycan content substantially equal to the glycosaminoglycan content of native cartilage tissue. Each of the layers comprises chondrogenic cells dispersed within an endogenously produced extracellular matrix.

According to another aspect of the present invention, a method is provided for generating a multilayer cartilage tissue construct. One step of the method comprises isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is then cultured in a serum-free culture medium for a time sufficient to permit the population of chondrogenic cells to differentiate and form a single layer cartilage tissue construct. The single layer cartilage tissue construct is removed from the bioreactor and formed into the multilayer cartilage tissue construct.

According to another aspect of the present invention, a multilayer cartilage tissue construct is produced by a method comprising the steps of isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is then cultured in a serum-free culture medium for a time sufficient to permit the population of chondrogenic cells to differentiate and form a single layer cartilage tissue construct. The single layer cartilage tissue construct is removed from the bioreactor and formed into the multilayer cartilage tissue construct.

According to another aspect of the present invention, a method is provided for generating a tracheal implant. One step of the method comprises isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is cultured in a serum-free culture medium for a time sufficient to permit the population of chondrogenic cells to differentiate and form a single layer cartilage tissue construct. The single layer cartilage tissue construct is removed from the bioreactor and the tracheal implant is formed with the single layer cartilage tissue construct.

According to another aspect of the present invention, a method is provided for repairing a tracheal cartilage defect in a subject. One step of the method comprises isolating a population of chondrogenic cells and then expanding the population of chondrogenic cells. Next, the population of chondrogenic cells is seeded into a bioreactor having a volume defined by oppositely disposed gas permeable membranes. The population of chondrogenic cells is cultured in a serum-free culture medium for a time sufficient to permit the population of chondrogenic cells to differentiate and form a single layer cartilage tissue construct. The single layer cartilage tissue construct is removed from the bioreactor and the tracheal implant is formed with the single layer cartilage tissue construct. Next, the tracheal cartilage defect is repaired with the tracheal cartilage implant.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a flowchart illustrating a method for generating a single layer cartilage tissue construct according to one aspect of the present invention;

FIG. 2 is a flowchart illustrating a method for generating a multilayer cartilage tissue construct according to another aspect of the present invention;

FIG. 3 is a flowchart illustrating a method for generating a tracheal implant according to another aspect of the present invention;

FIG. 4 is a flowchart illustrating a method for repairing a tracheal cartilage defect in a subject according to another aspect of the present invention;

FIG. 5 is a photograph showing a single layer cartilage tissue construct being applied to the surface of a titanium femoral condyle;

FIG. 6 is a photograph showing the single layer cartilage tissue construct of FIG. 5 covering the surfaces of both the medial and lateral condyles;

FIGS. 7A-B are histological cross-sections of single (FIG. 7A) and multilayered (FIG. 7B) cartilage tissue constructs. In FIG. 7A, the single layer cartilage tissue construct is 185 microns thick and filled with glycosaminoglycans (GAG) at a degree comparable to that of native cartilage. In FIG. 7B, the multilayer cartilage tissue construct has been formed by folding several layers on top of each other;

FIGS. 8A-B are histograms showing GAG and DNA assays of auricular chondrocytes pellets of a rabbit. For FIGS. 8A-B, the effect of dexamethasone removal from the culture after one week, and the effect of different culture times on the GAG and DNA production, were evaluated;

FIGS. 9A-J are histological cross-sections showing a comparison between sheets of cartilage formed by layering with (FIGS. 9B, 9D, 9F and 9H) and without (FIGS. 9A, 9C, 9E and 9G) compression. FIGS. 9A-D show toluidine blue staining of the layered lower sheet at lower (FIGS. 9A-B) and higher (FIGS. 9C-D) magnification. FIGS. 9E-I show collagen type II staining for the same samples shown above, confirming that all of the tissue produced in these layers is cartilage. FIG. 9I is the negative control for the antibody staining (no first antibody) and FIG. 9J shows an example of the material stained with saffranin O;

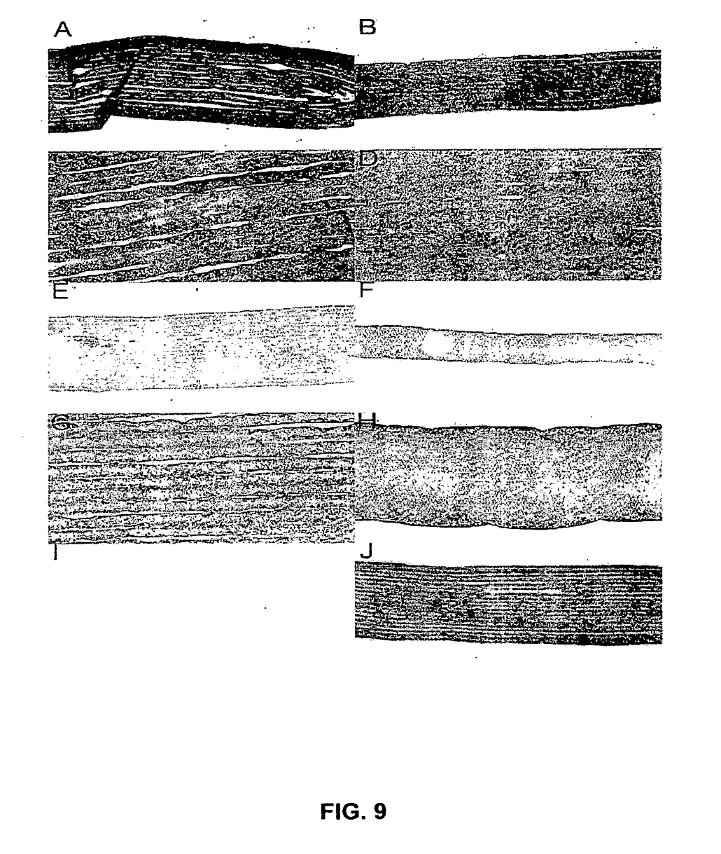

FIGS. 10A-C are histological cross-sections showing a single layer cartilage tissue construct prepared using different bioreactors;

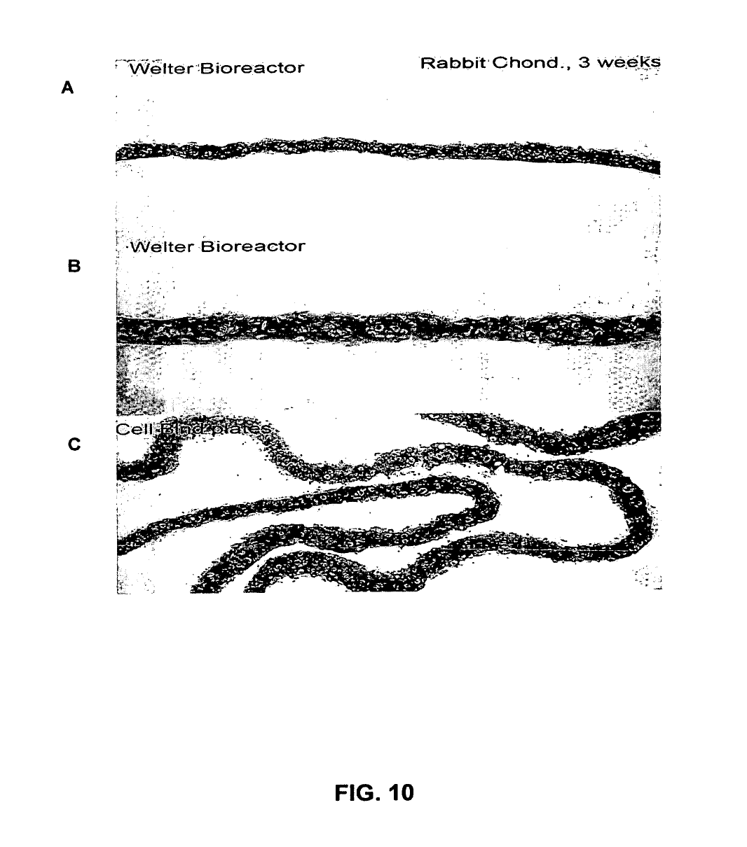

FIGS. 11A-B are histological cross-sections showing single layer cartilage tissue constructs after 3 weeks of culture;

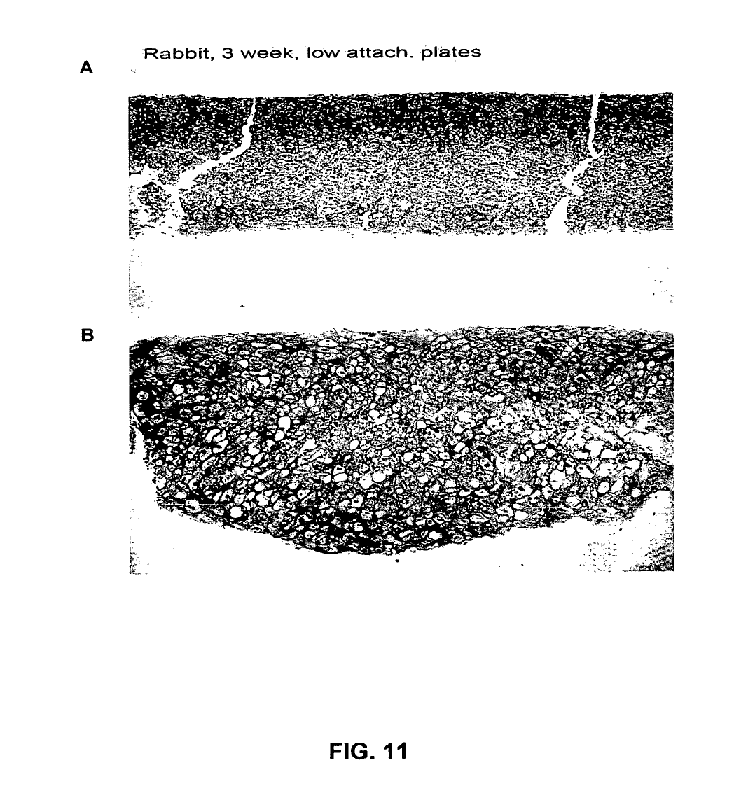

FIGS. 12A-B are photographs showing cartilage grafts. FIG. 12A shows a meshed skin graft wrapped around a silicone tube and secured with sutures. The epithelial side of the skin is facing inside. FIG. 12B shows an ear cartilage graft trimmed to the appropriate size. Oval holes were cut into the graft to allow transverse vascularization;

FIGS. 13A-C are photographs showing implantation of a neotrachea into a rabbit. FIG. 13A shows an exposed inferior superficial epigastrical artery in the lateral abdominal region and a raised pedicled fascial flap. FIG. 13B shows the fascial vascularized flap wrapped around the tube-skin graft construction. FIG. 13C shows a vascularized composite trachea consisting of a skin and cartilage graft. The silicone tube functions as a stabilizer (T=tube; CG=cartilage graft);

FIGS. 14A-B are photographs showing the neotrachea of FIG. 12A after implantation for 2 weeks. The neotrachea consists of a skin graft on the inside, a cartilage framework on the outside (made from auricular cartilage), and a vascularized fascial flap as an interface; and

FIGS. 15A-B are histological cross-sections of the neotrachea stained with H&E. FIG. 15A is at 1.25× magnification and FIG. 15B is at 10× magnification. After 16 days in vivo, the neotrachea has formed a stable and round-shaped framework (FIG. 15A) and all 3 layers appear healthy and integrated with some neovascularization (FIG. 15B) (*=epithelial graft; **=fascial flap; C=cartilage).

### DETAILED DESCRIPTION

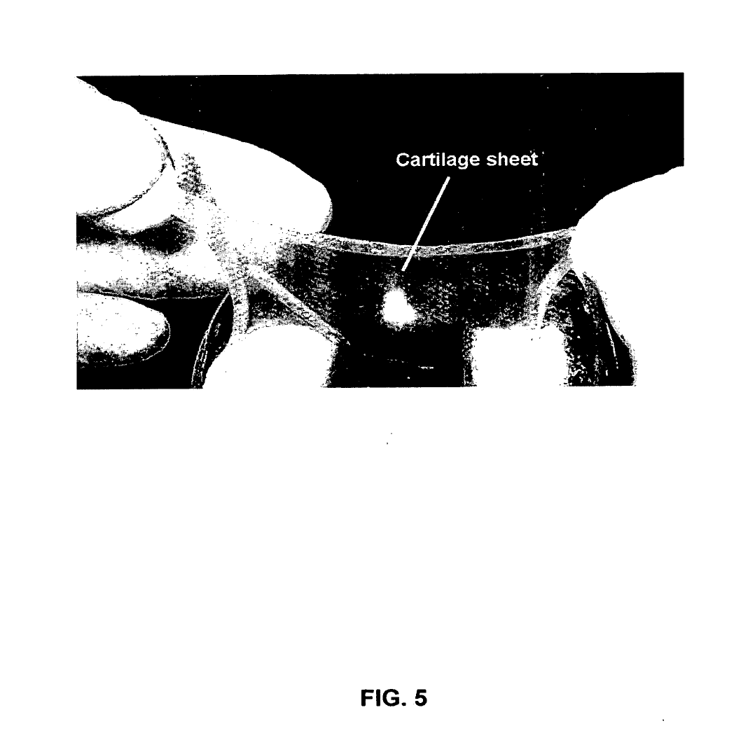

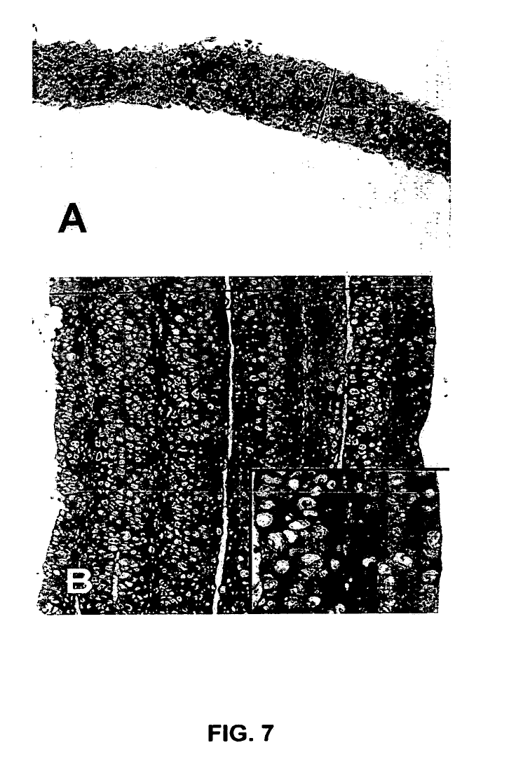

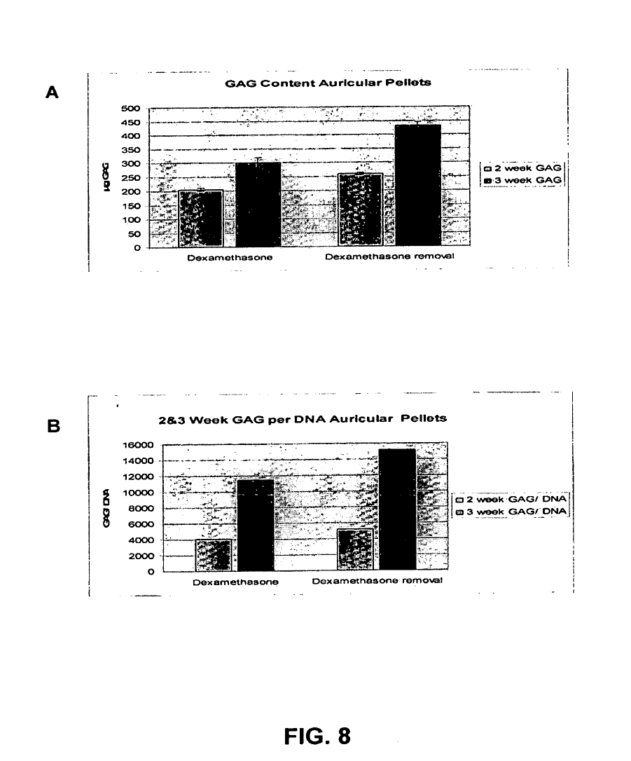

The present invention relates generally to methods and compositions for generating and repairing cartilage, and more particularly to methods and compositions for generating layered cartilage tissue constructs comprising chondrogenic cells dispersed within an endogenously produced extracellular matrix. The present invention is based on the discovery that culturing chondrogenic cells in a bioreactor with serum-free culture medium produces large, continuous sheets of cartilage (FIG. 5) with a glycosaminoglycan (GAG) content approximately equal to the GAG content of native cartilage (FIGS. 8A-B). The sheets of cartilage exhibit high tensile strength and can be layered upon one another to produce sheets of cartilage having an increased thickness (FIG. 7B). Based on this discovery, the present invention provides single and multilayer cartilage tissue constructs, methods for producing the single and multilayer cartilage tissue constructs, and therapeutic uses thereof.

All scientific and technical terms used in this application have meanings commonly used in the art unless otherwise specified. The definitions provided herein are to facilitate understanding of certain terms used frequently herein and are not meant to limit the scope of the present invention.

In the context of the present invention, the term “population” refers to an isolated culture comprising a homogenous, a substantially homogenous, or a heterogeneous culture of cells. Generally, a “population” may also be regarded as an “isolated” culture of cells.

As used herein, the term “chondrogenic cell” refers to any cell which, when exposed to appropriate stimuli, may differentiate and/or become capable of producing and secreting components characteristic of cartilage tissue.

As used herein, the term “cartilage” refers to a specialized type of dense connective tissue consisting of cells embedded in a matrix. There are several kinds of cartilage. Translucent cartilage having a homogeneous matrix containing collagenous fibers is found in articular cartilage, in costal cartilages, in the septum of the nose, in larynx and trachea. Articular cartilage is hyaline cartilage covering the articular surfaces of bones. Costal cartilage connects the true ribs and the sternum. Fibrous cartilage contains collagen fibers. Yellow cartilage is a network of elastic fibers holding cartilage cells which is primarily found in the epiglottis, the external ear, and the auditory tube. Cartilage is tissue made up of extracellular matrix primarily comprised of the organic compounds collagen, hyaluronic acid (a proteoglycan), and chondrocyte cells, which are responsible for cartilage production. Collagen, hyaluronic acid, and water entrapped within these organic matrix elements yield the unique elastic properties and strength of cartilage.

As used herein, the term “hyaline-like” refers to a type of cartilage known as hyaline cartilage. Hyaline cartilage includes the connective tissue covering the articular joint surface and may include, for example, articular cartilage, costal cartilage, and nose cartilage.

As used herein, the term “autogenic” refers to cells or tissues that are obtained from a donor and then re-implanted into the same donor.

As used herein, the term “allogenic” refers to cells or tissues that are obtained from a donor of one species and then used in a recipient of the same species.

As used herein, the term “construct” refers to a physical structure with mechanical properties, such as a matrix or scaffold.

As used herein, the term “mature chondrocyte” refers to a differentiated cell involved in cartilage formation and repair. Mature chondrocytes can include cells that are capable of expressing biochemical markers characteristic of mature chondrocytes, including, but not limited to, collagen type II, chondroitin sulfate, keratin sulfate, and characteristic morphologic markers including, but not limited to, rounded morphology observed in culture and in vitro generation of tissue or matrices with properties of cartilage.

As used herein, the term “immature chondrocyte” refers to any cell type capable of developing into a mature chondrocyte, such as a dedifferentiated or undifferentiated chondrocyte. Immature chondrocytes can include cells that are capable of expressing biochemical and cellular markers characteristic of immature chondrocytes, including, but not limited to, type I collagen, cathepsin B, modifications of the cytoskeleton, and formation of abundant secretory vesicles.

As used herein, the term “glycosaminoglycan” refers to long, unbranched polysaccharides consisting of repeating disaccharide units. Disaccharide units may consist of an N-acetyl-hexosamine and a hexose or hexuronic acid, either or both of which may be sulfated. Glycosaminoglycans can vary in the type of hexosamine, hexose or hexuronic acid unit they contain (e.g., glucuronic acid, iduronic acid, galactose, galactosamine, glucosamine). Glycosaminoglycans may also vary in the geometry of the glycosidic linkage.

As used herein, the term “tracheal cartilage defect” refers to any tracheal defect of, or injury to, the trachea. Tracheal cartilage defects may be caused by a variety of factors including, but not limited to, stenosis caused by implanted prosthetic devices, penetrating or blunt trauma, and tumors. Additionally, tracheal cartilage defects may be caused by congenital defects ranging from the complete absence of the trachea to an incomplete or malformed trachea.

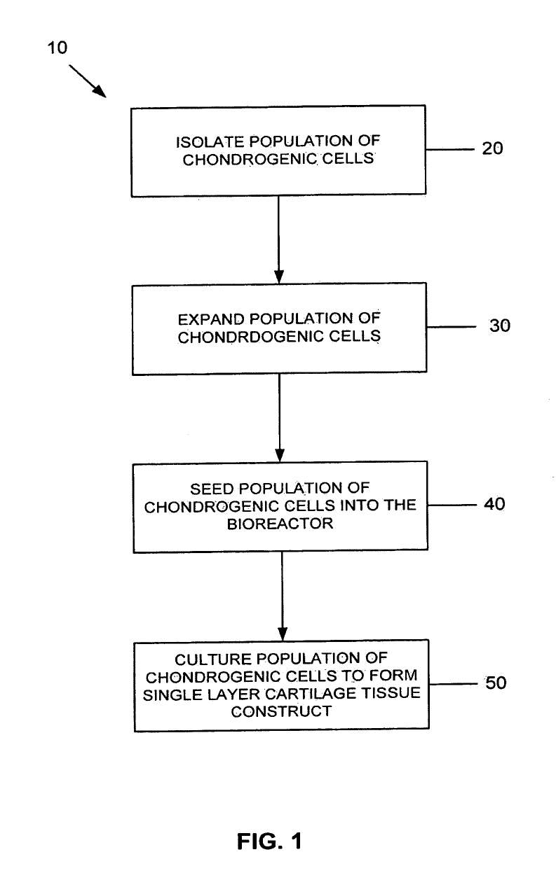

FIG. 1 is a flow diagram illustrating a method 10 for generating a single layer cartilage tissue construct in accordance with an aspect of the present invention. In the method 10, a population of chondrogenic cells may be isolated at 20. Chondrogenic cells may be isolated from essentially any tissue by obtaining, for example, a tissue biopsy. Chondrogenic cells may be isolated directly from pre-existing cartilage tissue such as hyaline cartilage, elastic cartilage, or fibrocartilage. More specifically, chondrogenic cells may be isolated from articular cartilage (from either weight-bearing or non-weight-bearing joints), costal cartilage, nasal cartilage, auricular cartilage, tracheal cartilage, epiglottic cartilage, thyroid cartilage, arytenoid cartilage, and/or cricoid cartilage. Alternatively, chondrogenic cells may be isolated from bone marrow.

Chondrogenic cells may be allogenic, autogenic, or a combination thereof, and may be obtained from various biological sources. Biological sources may include, for example, both human and non-human organisms. Non-human organisms contemplated by the present invention include primates, livestock animals (e.g., sheep, pigs, cows, horses, donkeys), laboratory test animals (e.g., mice, hamsters, rabbits, rats, guinea pigs), domestic companion animals (e.g., dogs, cats), birds (e.g., chicken, geese, ducks, and other poultry birds, game birds, emus, ostriches), captive wild or tamed animals (e.g., foxes, kangaroos, dingoes), reptiles and fish.

After obtaining a tissue biopsy of articular cartilage, for example, the chondrogenic cells may be released by contacting the tissue biopsy with at least one agent capable of dissociating the chondrogenic cells. Examples of suitable agents include trypsin and collagenase enzymes. As illustrated in Example 2 of the present invention, for example, a tissue biopsy may be sequentially digested in about 0.1% trypsin for about 30 minutes, about 0.1% testicular hyaluronidase for about 20 minutes, and about 0.1% collagenase type II for about 24 hours. The digestion may be carried out at about 37° C. in about a 20 ml volume. By digesting the tissue biopsy, a population of chondrogenic cells comprising mature chondrocytes, immature chondrocytes, or a combination thereof, may be successfully isolated from the tissue biopsy.

The isolated population of chondrogenic cells may next be expanded at 30 in a conditioned growth media effective to promote expansion of the cells. For example, once the chondrogenic cells have been isolated from the tissue biopsy, they may be proliferated ex vivo in monolayer culture using conventional techniques well known in the art. Briefly, the chondrogenic cells may be passaged after the cells have proliferated to such a density that they contact one another on the surface of a cell culture plate. During the passaging step, the cells may be released from the substratum. This may be performed by routinely pouring a solution containing a proteolytic enzyme, such as trypsin, onto the monolayer. The proteolytic enzyme hydrolyzes proteins which anchor the cells on the substratum and, as a result, the cells may be released from the surface of the substratum. The resulting cells may now be in suspension, diluted with culture medium, and re-plated into a new tissue culture dish at a cell density such that the cells do not contact one another. The cells subsequently re-attach onto the surface of the tissue culture and start to proliferate once again. Alternatively, the cells in suspension may be cryopreserved for subsequent use using techniques well known in the art.

In an example of the method 10, a population of immature chondrocytes may be expanded ex vivo in a conditioned growth media at a desired density. More particularly, the conditioned growth media may be prepared such that the cells are expanded under dedifferentiating conditions. As used herein, the term “dedifferentiating conditions” refers to culture conditions known to produce dedifferentiated cell phenotypes. As described in Example 2, for example, about 10 ml of calf serum may be added to the cell suspension following digestion. The cell suspension may then be passed through a 70 μm Nitex filter, centrifuged, and plated out in DMEM containing about 10% fetal bovine serum. The medium may be replaced twice per week, and confluent plates may be passaged as needed to obtain the desired cell density.

After the population of chondrogenic cells has been expanded to the desired density, the cells may be seeded or loaded into a culture vessel at 40. The culture vessel may comprise a bioreactor, such as an OptiCell™ (BioCrystal Ltd., Westerville, Ohio) bioreactor, having a volume defined by oppositely disposed gas permeable membranes. The gas permeable membranes allow the chondrogenic cells to have continuous access to nutrients while also allowing diffusion of waste products from the vicinity of the cells. More specifically, the chondrogenic cells may be seeded into the bioreactor at a desired density. For example, the cells may be seeded into the OptiCell™ bioreactor at a density of, for example, about 1×10⁵ cells/mL to about 100×10⁶ cells/mL.

It should be understood that seeding the cells into the OptiCell™ bioreactor at a higher density is preferred because doing so encourages the cells to switch from a growth phase to a differentiation phase and produce high levels of endogenous extracellular matrix. The term “endogenous extracellular matrix” as used herein refers to a chondrogenic cell-derived structural substance produced in vitro. The extracellular matrix provides a growth template for the cells to grow, differentiate, and bind together into a single cartilage tissue construct.

Once the chondrogenic cells have been seeded into the OptiCell™ bioreactor, the cells may be cultured in a serum-free, defined chondrogenic medium at 50. The serum-free medium may comprise, for example, high-glucose DMEM supplemented with dexamethasone, ascorbate-2-phosphate, sodium pyruvate, and a premix of insulin, transferring and selenium (ITS). More particularly, and as described in Examples 1 and 2, the serum-free medium may comprise high-glucose DMEM containing about 100 mM sodium pyruvate, about 80 μM ascorbate-2-phosphate, about 100 nM dexamethasone, and about 1% ITS. Additional serum-free medium components may include L-Glutamine, MEM non-essential amino acid solution, and/or an antibiotic/antimycotic.

It should be appreciated that growth factors may also be added to the serum-free medium to enhance or stimulate cell growth. Examples of growth factors include, but are not limited to, transforming growth factor-β, platelet-derived growth factor, insulin-like growth factor, acid fibroblast growth factor, basic fibroblast growth factor, epidermal growth factor, hepatocytic growth factor, keratinocyte growth factor, and bone morphogenic protein. It should also be appreciated that other agents, such as cytokines, hormones (e.g., parathyroid hormone, parathyroid hormone-related protein, hydrocortisone, thyroxine, insulin) and/or vitamins (e.g., vitamin D) may also be added or removed from the serum-free medium to promote cell growth.

After the chondrogenic cells have been seeded into the OptiCell™ bioreactor, the cells may be cultured under appropriate conditions. For example, the cells may be cultured at about 37° C. in about a 5% carbon dioxide atmosphere at about 90% to about 95% humidity. The oxygen percentage can be varied from about 1% to about 21%. The cells may be cultured for about 1 to about 3 weeks in the OptiCell™ bioreactor.

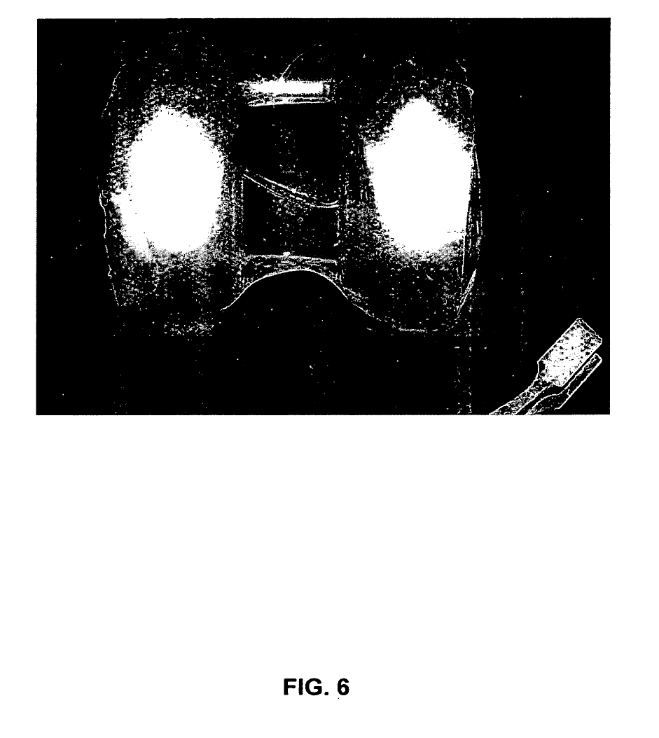

After culturing the cells for an appropriate amount of time, the single layer cartilage tissue construct may be harvested by cutting away the gas permeable membrane (e.g., with a scalpel) and lifting the single layer cartilage tissue construct out of the OptiCell™ bioreactor. The single layer cartilage tissue construct may then be applied, for example, to an articular surface as shown in FIGS. 5 and 6. The single layer cartilage tissue construct may be attached to the articular surface using, for example, adhesive materials such as bioadhesives, sutures, and/or staples at the edges of the articular surface. Examples of adhesive materials can include, but are not limited to, calcium phosphate-based pastes (e.g., αBMM), fibrin-based glues, transglutaminase, and chemical cross-linking agents (e.g., 1-ethyl-3-[3-dimethylaminopropyl]carbodiimide hydrochloride).

Where the single layer cartilage tissue construct is applied to an articular surface, the articular surface may be first prepared by removing all loose fibrillar material, along with any remaining cartilage, down to either the calcified cartilage layer or the bone. It is desirable to avoid any bleeding or access to bone marrow in order to minimize interference with the adhesive used to fix the single layer cartilage tissue construct in place. Depending upon the clinical needs of the subject, however, the bone marrow space may be purposefully accessed in order to promote an osteogenic response at the graft-host interface to facilitate integration of the single layer cartilage tissue construct into the native cartilage. The adhesive material may be spread on the articular surface, the surface of the single layer cartilage tissue construct to be attached, or both. Additionally or optionally, the single layer cartilage tissue construct may be positioned over the entire articular surface and the adhesive material sutured onto the joint using either staples or sutures (e.g., resorbable sutures).

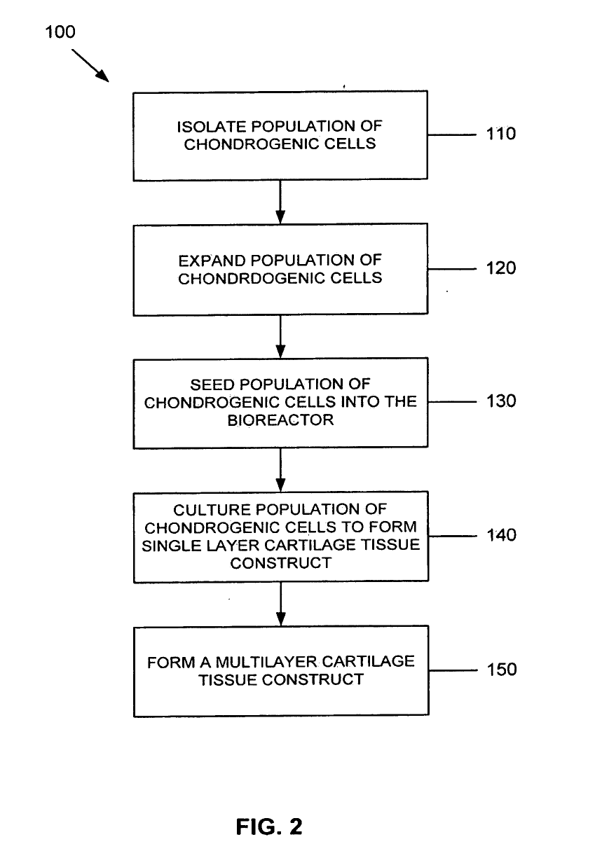

FIG. 2 is a flow diagram illustrating a method 100 for generating a multilayer cartilage tissue construct in accordance with another aspect of the present invention. In the method 100, a population of chondrogenic cells may be isolated at 110 and expanded ex vivo at 120 as described above. As also described above, the chondrogenic cells may then be seeded into a bioreactor at 130 and cultured to form a single layer cartilage tissue construct at 140.

After the single layer cartilage tissue construct is formed at 40, a multilayer cartilage tissue construct may then be formed at 150 using a variety of techniques. According to one technique, a first portion of the single layer cartilage tissue construct may be folded over onto a second portion of the single layer cartilage tissue construct and then placed in a culture vessel for an appropriate amount of time. For example, one half of the single layer cartilage tissue construct may be folded over onto the other half of the single layer cartilage tissue construct and then placed in a culture flask for about 1 week. The hydrostatic pressure exerted by the growth medium on the folded portions of the single layer cartilage tissue construct promotes integration of the layers and formation of a multilayer cartilage tissue construct.

According to another technique for forming the multilayer cartilage tissue construct, at least two single layer cartilage tissue constructs may be layered on top of one another and then placed in a culture vessel under a compressive load. For example, about 10 to 15 single layer cartilage tissue constructs may be layered on top of each other and then placed under static compression for about 1 week.

The device for applying the compressive load should be non-toxic and sterile. The device should also be permeable to liquid and gas diffusion, composed of a discontinuous layer, such as a grid, and should be used transiently to allow for nutrient exchange within the tissue. Enough force should be applied to the device to ensure contact between the layers while also not causing significant cell death. The compression may be continuous or cyclical and may be of variable duration depending upon the growth and synthesis characteristics of the particular set of chondrogenic cells being used. The compression can be modified by altering the mechanical load, the duration of the load, the magnitude of the load, making the load cyclical, altering the number, duration, and force during each cycle, and by altering the amount of strain placed on the layers during application of the force.

After forming the multilayer cartilage tissue construct, the multilayer cartilage tissue construct may be removed from the culture vessel and applied to an articular surface, for example, as described above. It should be appreciated that a combination of hydrostatic and mechanical pressure may be used to form the multilayer cartilage tissue construct. Additionally, it should be appreciated that the thickness of the multilayer cartilage tissue construct may be adjusted as needed by adding or removing layers. For example, a plurality of layers may be annealed and formed into a multilayer cartilage tissue construct having a thickness of about 1 mm to about 4 mm or greater.

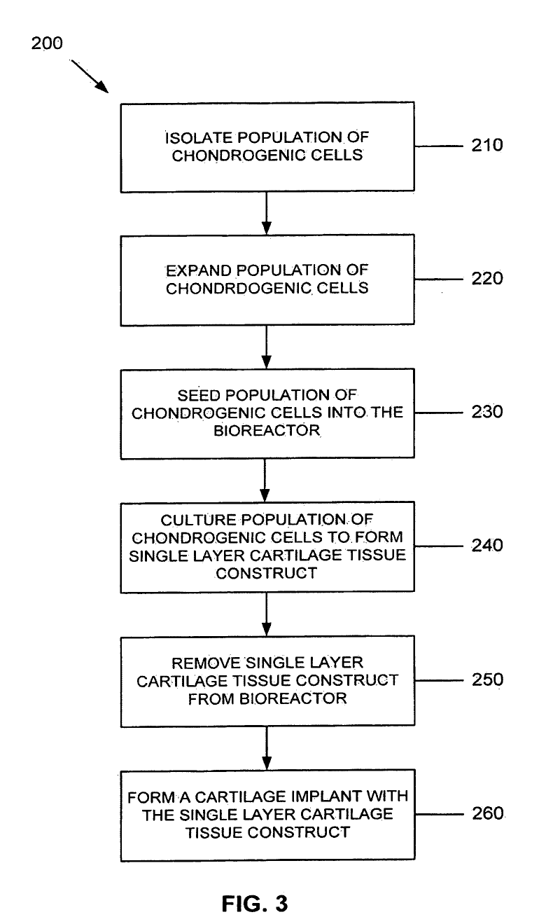

FIG. 3 is a flow diagram illustrating a method 200 for generating a tracheal implant in accordance with another aspect of the present invention. In the method 200, a population of chondrogenic cells may be isolated at 210 and expanded at 220 as described above. As also described above, the population of chondrogenic cells may then be seeded into a bioreactor at 230 and cultured to form a single layer cartilage tissue construct at 240. After forming the single layer cartilage tissue construct, the single layer cartilage tissue construct may be removed from the bioreactor at 250.

At 260, the tracheal implant may be formed from of a single layer cartilage tissue construct or, alternatively, from a multilayer cartilage tissue construct. Depending upon the clinical needs of the subject, a multilayer cartilage tissue construct may be used to form a whole trachea or only a portion of a whole trachea. For example, a tracheal implant comprising a whole trachea may be formed by first obtaining a tube-shaped tracheal construct comprised of, for example, a biocompatible and/or bioresorbable material. The tracheal construct may be optimally sized to suit the needs of the subject. A single or multilayer cartilage tissue construct may then be wrapped around the tracheal construct and secured with a fibrin sealant and/or sutures, for example. The fibrin glue may be applied between the layers to prevent shear movement and to promote integration of the layers. After the tracheal implant has been formed, the implant may be used to repair a tracheal cartilage defect as described in greater detail below.

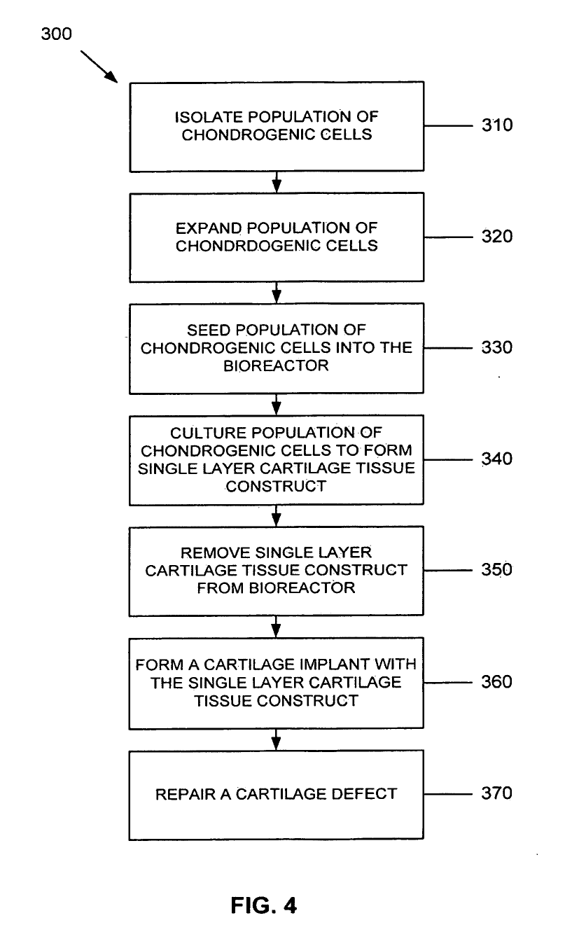

FIG. 4 is a flow diagram illustrating a method 300 for repairing a tracheal cartilage defect in a subject in accordance with another aspect of the present invention. In the method 300, a population of chondrogenic cells may be isolated at 310 and expanded at 320 as described above. As also described above, the population of chondrogenic cells may then be seeded into a bioreactor at 330 and cultured to form a single layer cartilage tissue construct at 340. After forming the single layer cartilage tissue construct, the single layer cartilage tissue construct may be removed from the bioreactor at 350 and then formed into a tracheal implant at 360 as described above.

At 370, repair of a tracheal cartilage defect may begin by first identifying the defect. Tracheal cartilage defects may be readily identifiable by visually identifying the defects during open surgery of the trachea or, alternatively, by using computer aided tomography, X-ray examination, magnetic resonance imaging, analysis of serum markers, or by any other procedures known in the art.

Once the tracheal cartilage defect has been identified, an appropriately sized tracheal implant may be selected. For example, the tracheal implant may have a size and shape such that when the tracheal implant is implanted, the edges of the tracheal implant directly contact the edges of native cartilage tissue. The tracheal implant may be fixed in place by, for example, surgically fixing the patch with bioresorbable sutures. Additionally or optionally, the tracheal implant may be fixed in place by applying a bioadhesive to the region interfacing the tracheal implant and the tracheal cartilage defect. Examples of suitable bioadhesives include fibrin-thrombin glues and synthetic bioadhesives similar to those disclosed in U.S. Patent No. 5,197,973.

In one example of the method 300, the cartilage tissue defect may comprise a stenotic portion of the trachea, such as two of the cartilages comprising the trachea, caused by prolonged placement of a tracheal T-tube. To repair the tracheal cartilage defect, the stenotic portion may first be surgically excised. Next, a tracheal implant may be formed having a size and shape complementary to the size and shape of the excised stenotic portion. The tracheal implant may then be surgically fixed in place of the excised stenotic portion by an end-to-end anastomosis. After the tracheal implant has been suitably fixed in place, the surgical procedure may be completed and the tracheal implant permitted to integrate into the native cartilage tissue.

In an alternative example of the method 300, the tracheal cartilage defect may comprise a congenital defect, such as a missing trachea, in a pediatric subject. A tracheal implant comprising a whole trachea may be prepared and then surgically implanted into the subject by an end-to-end anastomosis. After the tracheal implant has been suitably fixed in place, the surgical procedure may be completed and the tracheal implant permitted to integrate into the native tissue. By providing the subject with a whole tracheal implant, the tracheal implant may integrate into the native tissue and grow along with the subject, thus removing the need to perform additional surgeries as the subject ages.

The following examples are for the purpose of illustration only and are not intended to limit the scope of the claims, which are appended hereto.

### EXAMPLE 1

### Introduction

Currently, there is no adequate treatment for large tracheal defects. The standard treatment for isolated tracheal stenoses is a segmental tracheal resection, followed by an end-to-end anastomosis. However, segmental resection is not possible in long-segmented stenosis (>50% of total tracheal length or more than 6.0 cm length of stenosis) and the reconstruction of large tracheal defects remains an unsolved challenge for surgeons.

Many attempts have been made to produce tracheal prostheses, for example, through the use of a non-autologous tracheal graft. Non-degradable prostheses have also been tested, but never reached successful clinical application, because of their limited success due to immune rejection, graft ischemia or infections. Internal stents have also been used as primary treatment modalities for benign tracheal stenosis. The purpose of a stent is to counteract contraction of scar tissue and to maintain an open airway. However, disadvantages of internal stenting include biocompatibility problems, accidental displacement, and lack of tracheal mucociliary clearance with the tendency to accumulate secretions in the lumen causing airway obstruction.

### Clinical Significance

Chemical injury, tumours, and inflammatory diseases are possible causes of tracheal stenoses. However, long-term intubation and tracheotomy are by far the most common reasons. Despite great advances in the intensive care of ventilated patients, the incidence of tracheal stenoses following long-term intubation or tracheotomy remains up to 21%.

Usually the pressure of the inflated cuff is responsible for the damage of mucosa and cartilage, which can lead to inflammation and scarring resulting in a tracheal stenosis. Tracheal stenoses of 30% or more usually become symptomatic and require intervention. Patients with severe tracheal stenoses require a tracheostoma for breathing, which almost always leads to a loss of vocal function. This is especially true for stroke patients with paralyses who depend on their voice as their only means of communication. In infants between the age of 1-4 years, an existing tracheostoma can lead to an impaired development of the speech, which may ultimately result in psycho-social deficits.

The treatment of choice for symptomatic benign tracheal stenoses is therefore a segmental tracheal resection, followed by a primary end-to-end anastomosis. The success rate of this procedure is high and lies between 77% and 97%. In contrast, segmental tracheal resection is not recommended for long-segmented stenoses (>50% of total tracheal length or more than 6 cm length of stenosis), since it is not possible to establish a tension-free end-to-end anastomosis, which can possibly result in severe complications, such as anastomotic rupture or re-stenosis. In these cases, a re-tracheostomy is very often inevitable and the only possible treatment option. In such cases, a substitute for the native trachea would be an ideal solution.

The trachea is a very complex organ consisting of an outer cartilaginous framework, which is rigid enough to give support and keep the airway open but simultaneously flexible to allow head movements. Internally, the trachea is lined with a ciliated mucosa, which has a clearance function for the mucous. In addition, both the epithelium and the cartilage are dependent on layers of vascularized support tissue. Due to its critical function, it is very difficult to build a substitute that meets all the following characteristics: an airtight lumen with lateral rigidity and vertical elasticity; a continuous internal lining; a reliable healing tendency; good vascularization; and the ability to integrate into neighbouring tissue.

In view of this, Rose et al. were the first to report on a homologous tracheal transplantation, and Herberhold et al. performed several successful homologous tracheal transplantations in children. However, due to the high failure rate, tracheal transplantation never became a viable treatment. Other authors focused on using various types of prostheses for tracheal replacement with limited success because of graft ischemia, immune rejection, poor epithelization and chronic infection.

In respect to these difficulties, attempts have been made to develop an autologous tracheal graft, consisting of a cartilage framework and an inner mucosal lining. However, their uptake is poor, if used as free grafts and therefore various attempts have been made to achieve vascularization of autologous grafts. Prefabrication methods offer the ability to create a vascularized graft, which can be used for reconstruction. Different vascular flaps have been tested in animal models for tracheal reconstruction. In each of these studies autologous tissue was combined with some kind of artificial material. In addition, in some of the studies no internal lining was created for the tracheal graft. Lykoudis et al. developed an axial prefabricated vascular composite graft using epidermis from the ear as an internal lining. However, in that study, they used a bio-synthetic vascular graft for reinforcement, which can potentially cause biocompatibility problems, chronic inflammation or can be subject to rejection. In this context, the ideal tracheal graft material is a well vascularized composite graft consisting of autologous tissue that is not subject to rejection. Unfortunately, the amount of cartilage in the body that can be harvested for reconstruction is very limited.

Tissue engineering has the potential to overcome these difficulties. The primary goal of cartilage tissue engineering is to overcome the poor ability for repair by promoting the mitotic and differentiation potential of autologous chondrocytes to replace damaged tissue. To achieve this goal it is necessary to develop methods to harvest and expand chondrocytes and to engineer the cartilage repair tissue of the required size for reconstruction. Secondly, the engineered cartilage must meet certain biomechanical requirements in order to function as a tracheal framework. Tissue engineering is gaining increasing acceptance in cardiothoracic surgery and represents the most promising technique to create a functional trachea.

### Preliminary Studies

Engineering Cartilage Sheets

Four New Zealand White male rabbits, 3.0-3.5 kg of weight, were used to harvest a 5×5 mm piece of ear cartilage. The cartilage was dissected free of perichondrial tissues, cut into 1×1 mm pieces, and digested enzymatically. The chondrocyte cell suspension was plated out in Dulbecco's Modified Eagle's Medium (DMEM) containing 10% fetal bovine serum. Medium was exchanged twice weekly, and confluent plates were passaged by standard methods using trypsin. Chondrocytes from first and second passage were used to form engineered cartilage tissue. The expanded cells were loaded into a 2 mm thick cell culture chamber with 2 gas permeable membranes on either side of the tissue culture chamber at high density in chondrogenic defined medium. Three different cell loading densities were tested: 30×10⁶, 50×10⁶, and 100×10⁶ cells per chamber. The medium consisted of Dulbecco's Minimal Essential Medium containing 100 mM sodium pyruvate, 80 micromolar ascorbate-2-phosphate, 100 nM dexamethasone (dex) and a premix of insulin, transferrin and selenium (1% ITS, Becton Dickinson Labware). Medium was replaced 3 times per week. By delivering culture-expanded cells into the chamber at high density, the chondrogenic cells immediately switched from a growth phase to a differentiation phase and produced high levels of extracellular matrix. When loading the chamber system with 30×10⁶ cells, the extracellular matrix bound the cells together into one large sheet which was then lifted off the original attachment surface. The floating sheet was inhibited from folding by being constrained between two sheets of air-permeable material spaced approximately 2 mm apart. When loading the camber with higher densities (100×10⁶ cells), the media became acidic and the cells clumped up and did not form any cartilage sheets. Chambers loaded with 30 and 50 million cells remained healthy and produced sheets of cartilage. As expected, the chambers with 50 million cells produced thicker sheets, so additional studies will be conducted to upwardly adjust this number without detracting from cell viability or matrix production.

The sheets were cultured for 3 weeks and then harvested by cutting away the gas permeable sheet with a scalpel and lifting the entire cartilage sheet out. The cartilage material proved to have enough strength to be handled under mild tension, as is shown in FIG. 5 where a cartilage sheet is applied to the surface of a titanium femoral condyle implant to demonstrate its potential use in orthopedic applications.

All in vitro formed cartilage sheets were prepared for histologic examination. The cartilage material was fixed, embedded in paraffin, and sectioned to determine its size along with its cellular and matrix makeup (FIGS. 7A-B). One of the limitations of the cartilage fabrication process is that the cartilage thickness is only about 150-300 microns in thickness. In order to increase the thickness of the implant material, the cartilage was folded upon itself and placed in culture. FIG. 7B shows the results of this layering technique in which several cartilage layers have been placed upon each other and incubated in culture for a week. After one week, histologic examination revealed a set of integrated layers with an uninterrupted matrix spanning some, but not all of the stacked layers (FIG. 7B).

Effect Of Removal of Dexamethasone and Culture Time on Chondrocyte Pellets

Another method to increase cartilage thickness is to promote a more robust production of matrix material by changing culture conditions. It has been reported that dex induces chondrogenic differentiation and chondrogenesis. It has also been shown that supplementation of serum free media with dexamethasone increases the glycosaminoglycan content of chondrogenic pellets of human articular cartilage. It was hypothesized that the timing of dex exposure was another important factor in the chondrogenesis differentiation sequence and that long-term exposure to dex might be detrimental. To determine the effect of removing dex from pellets made from auricular chondrocytes of rabbits, the pellets were grown in Falcon tubes in a defined culture media which consisted of Dulbecco's Minimal Essential Medium containing 100 mM sodium pyruvate, 80 micromolar ascorbate-2-phosphate, 100 nM dex and a premix of insulin, transferrin and selenium (1% ITS, Becton Dickinson Labware). After the first week, dex was removed from the culture media in one group, where as the second group was continued on dex. Each of the 2 groups were further split into 2 groups, one culturing 2 and the other for 3 weeks. Each of the 4 groups consisted of samples:
Group 1: defined media was given for 2 weeks; dex was given for the first week and then discontinued
Group 2: defined media was given for 3 weeks; dex was given for the first week and then discontinued
Group 3: defined media, including dex, was given for 2 weeks
Group 4: defined media, including dex, was given for 3 weeks

At each time point (2 and 3 weeks) the pellets were collected and a GAG and DNA assay was performed in each pellet. The data showed that removal of dex led to an increased GAG level of 26% after 2 weeks and 45% after 3 weeks (FIG. 8B). The data also indicated that the GAG amount of the pellets increases significantly between the week 2 and 3 in culture: within the group, which received dex the entire time in culture, there was a 47% increase, whereas the dex-removal-group demonstrated a 67% increase. In summary, the GAG content of auricular chondrocyte pellets raises significantly (all results comparing dex and dex removal data were statistically significant at p<0.05 by Student's T-test) when culturing for 3 weeks instead of 2 and when removing dex from the culture media after the first week.

### Fabrication of a Neo-Trachea in a Rabbit Model

A total of 3 New Zealand White rabbits, weighing between 3.0 kg and 3.5 kg, were used for creating a vascularized neo-trachea. The rabbits were anesthetized by intramuscular injection of ketamine hydrochloride and xylazine. All procedures were performed under sterile conditions.

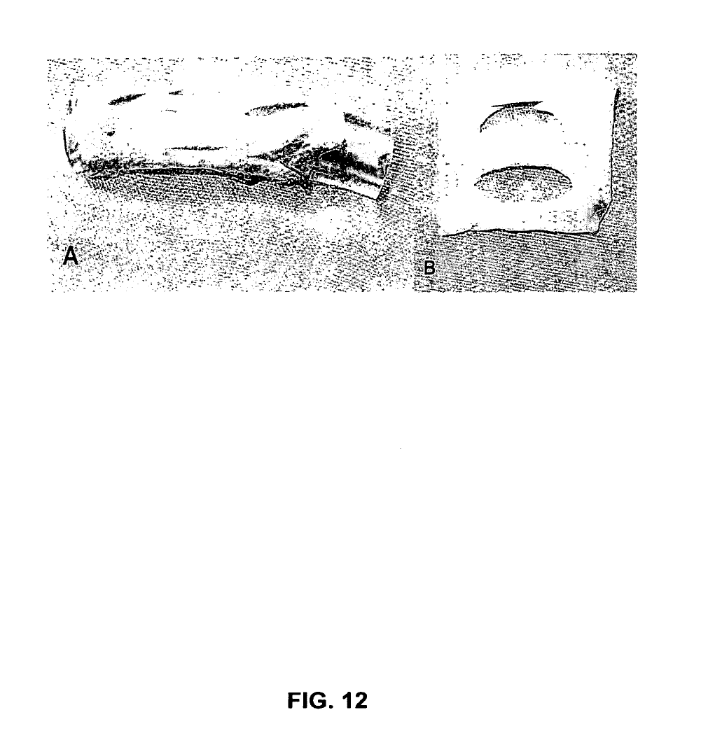

A 2×2 cm piece of full thickness skin from the inner side of the base of the ear as well as a 2×3 cm ear cartilage piece were harvested. The skin was completely defatted and meshed. It was then wrapped around a 2.5 cm long silicone tube with a diameter of 8 mm (FIG. 12A), the epithelial side facing the stent, and secured with 5-0 vicryl sutures. The cartilage was trimmed to the appropriate size and shape and oval holes were cut into the cartilage graft to allow transverse vascularization (FIG. 12B).

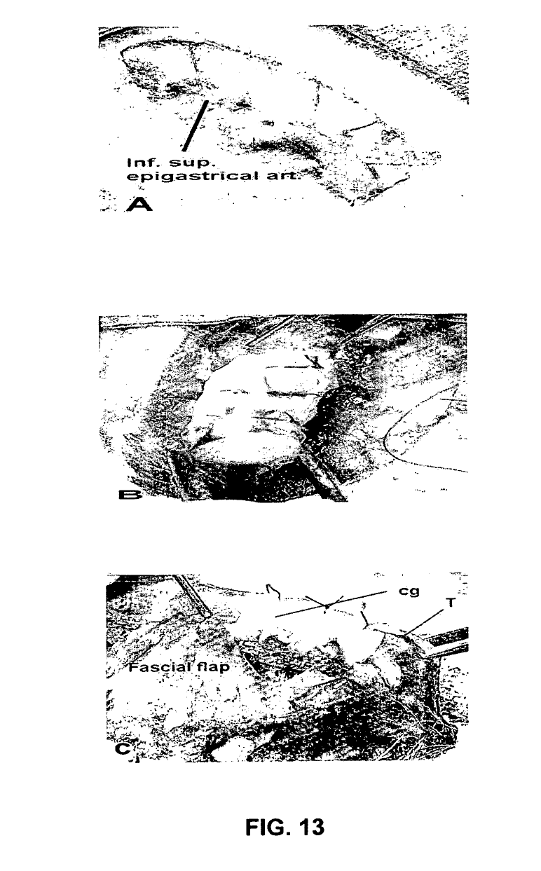

A 4 cm vertical skin incision was then made in the rabbit's lateral abdominal area. The inferior superficial epigastrical artery was identified and a fascial flap, based to the artery, was raised (FIG. 13A). The silicone tube with the epithelium was implanted and a thin layer of fascia was wrapped around, which was pedicled on both ends, providing the construct with a sufficient blood supply (FIGS. 13B-C). Then, the cartilage graft was wrapped around and secured with several 5-0 vicryl sutures. The construct was then buried in a fascial pocket under the skin, followed by the wound closure.

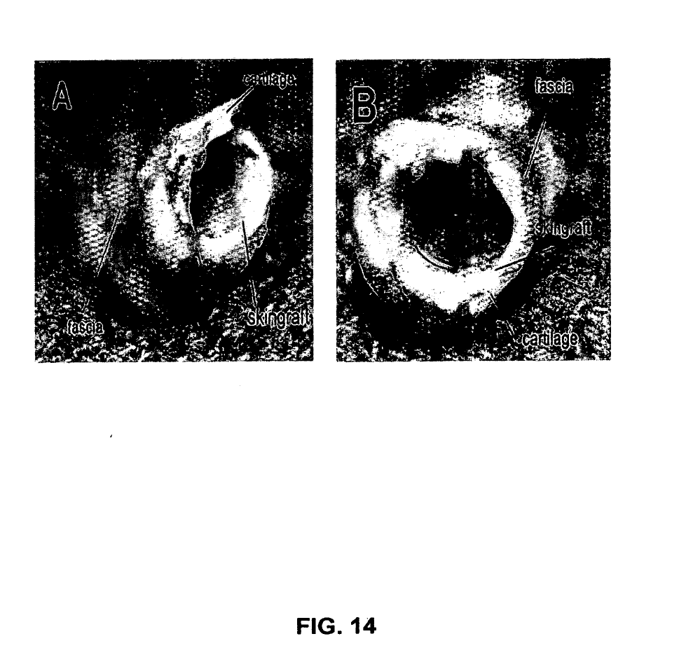

After 18 days, the animals were sacrificed and the tracheal construct harvested. The postoperative course had been uneventful. Macroscopically, there were no signs of infection. The neo-trachea was pink and healthy without signs of a compromised blood supply. After removing the tube, the neo-trachea maintained its round shape and histologic cross sections demonstrated that all layers were integrated well (FIGS. 14A-B).

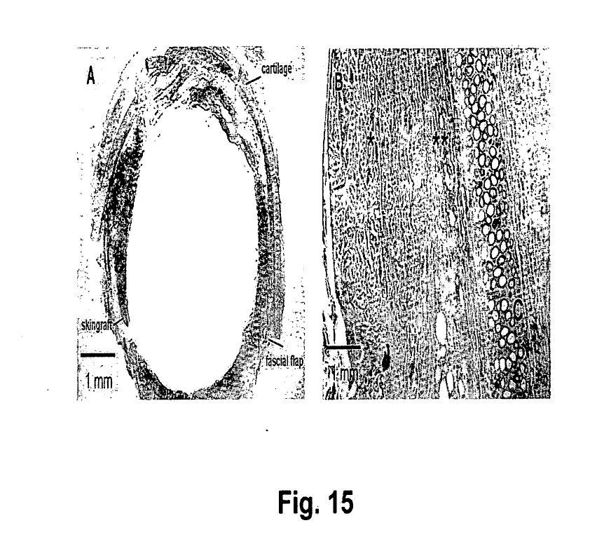

The meshed epithelium had formed an intact inner lining without any visible hair. The histological analysis of the neo-trachea revealed an excellent integration of all 3 layers. Epithelium, fascial flap and the cartilage were tightly attached to each other. There were no signs of inflammation or cartilage degradation (FIGS. 15A-B).

### EXAMPLE 2

### Introduction

The goal of this experimentation is to determine if the increase in matrix production observed in aggregate cultures that go through a dex removal process translates into matrix increase in the bioreactor system. In addition, it has been shown that TGF-β1 not only has a positive influence on chondrocyte proliferation but also enhances mesenchymal stem cell redifferentiation capacity in aggregate cultures. Therefore, TGF-β1 is one of the most likely candidates for promoting matrix production. Many authors report that the GAG concentration is well correlated with a measure of mechanical stiffness of the cartilage. With increasing biomechanical properties of the sheets, it is assumed that they give better support as a cartilage framework for the neo-trachea. Different culture conditions are tested to optimize the process of forming strong cartilage sheets. Dex is usually a supplement to the culture media for chondrocyte pellets, since it has redifferentiating effects on the cells. On human stem cells, dex has chondrogenic effects and is required for chondrogenic differentiation. The preliminary data showed that the removal of dex from the culture media after the first week significantly increased the GAG/DNA content of auricular chondrocyte pellets.

### Experimental Plan

The effects of dex removal and TGF-β1 supplementation is evaluated using identical analytic methods, only the culture conditions are different. For both, the dex and TGF-β1 arms of the experiment, 2 groups are examined—the control and the experimental groups—each containing of 6 samples. For the dex removal experiments, dex is added as a supplement at a concentration of 10⁻⁷M, as usual. In one group, dex is discontinued after 1 week, whereas the second group receives dex throughout the 3 weeks. For the TGF-β1 arm of the study, there are two groups, each consisting of 6 samples, with one receiving TGF-β1 as a supplement at a concentration of 10 ng/ml while the control group receives no supplementation. All cartilage sheets (Dex group, TGF-β1 group, and control groups) are cultured in the bioreactor for 3 weeks and then evaluated as described below.

To evaluate the effects of TGF-β1 and the effects of removing dex from the culture media, all cartilage sheets are analyzed histologically as well as by immunochemistry and biochemistry. In addition, dGEMRIC analysis of the cartilage sheets is performed, which allows a quantitative assessment of the cartilage GAG content and a 3-dimensional image of the GAG distribution of the cartilage sheet can be created. dGEMRIC utilizes a charged contrast agent (Gd-DTPA²⁻) that distributes in cartilage in inverse proportion to the negative charged GAG's. This is a novel method, which has been evaluated against biochemical and histological measures. The sheets are harvested after 3 weeks and prepared for imaging studies, histology, immunostaining and biochemistry. After the cartilage sheet has been removed from the chamber system it has a size of approximately 6×6 cm. Each half of the sheet is cut in 6 even quadrants. The 6 quadrants of one half are used for dGEMRIC, whereas a punch is used for the other 6 quadrants to cut 3 defined round pieces. In total, there will be 18 round samples: 6 for histological, 6 for immunochemical and 6 for biochemical testing.

The number of samples needed is determined with an on-line (http://home.clara.net/sisa/) sample size analysis program using the GAG assay results, where the largest standard deviation is 6.2% of the mean. The calculation is based on a double sided T-test analysis with a power of 0.90 and an alpha of 0.01, showing that 3 samples is sufficient to detect a 20% difference between means. However, since the GAG analysis standard deviations do not reflect animal to animal differences, the number of samples needed is doubled.

Twelve male New Zealand White rabbits, which are between 12 and 14 months old, are used for this study. Samples with or without TGF-β1 treatment or removal of dex are compared, statistically, using a double sided Student's T-test. Based on estimates from the preliminary data, using 6 samples, it is possible to detect 10% differences in the means at a power of 0.80 and an alpha of 0.05. Since we are only interested in relatively large differences, 20% or greater, this is a sufficient sample size for this analysis.

Samples with or without TGF-β1 treatment or removal of dex are compared, statistically, using a double sided Student's T-test. Based on estimates from the preliminary data, using 6 samples, it is possible to detect 10% differences in the means at a power of 0.80 and an alpha of 0.05. Since we are only interested in relatively large differences, 20% or greater, this is a sufficient sample size for this analysis.

Chondrocyte Culture

Auricular cartilage is harvested from each animal by removing a 5×5 mm cartilage piece under sterile conditions while the animal is under general anaesthesia. The cartilage piece is placed in sterile saline solution for further processing in a sterile culture hood. The cartilage is dissected free of perichondrium on either side, the cartilage tissue cut into approximately 1×1 mm pieces, and sequentially digested in 0.1% trypsin (30 min), 0.1% testicular hyaluronidase (20 min), and 0.1% collagenase type II (24 hr). All digestions are carried out at 37° C. in a 20 ml volume. Following collagenase digestion, 10 ml of calf serum is added and the cell suspension is passed through a 70 μm Nitex filter, centrifuged and plated out in Dulbecco's Modified Eagle's Medium (DMEM), containing 10% fetal bovine serum. Medium is replaced twice per week, and confluent plates are passaged by standard methods using trypsin. Chondrocytes from first and second passage are used to form engineered cartilage sheets. The expanded cells are loaded into a cell culture chamber with a 2 mm thick chamber with 2 gas permeable membranes on either side of the tissue culture chamber at density of 30×10⁶ cells in chondrogenic defined medium. The medium consists of Dulbecco's Minimal Essential Medium containing 100 mM sodium pyruvate, 80 micromolar ascorbate-2-phosphate, 100 nM dex and a premix of insulin, transferrin and selenium (1% ITS, Becton Dickinson Labware). Medium is changed 3 times per week using the chamber ports. Culture conditions are at 37° C. in a 5% carbon dioxide atmosphere at 90-95% humidity and atmospheric oxygen (21%).

Histology/Immunochemistry

Collagen Type I is a structural element and mainly expressed in fibrous connective tissue, Type II collagen represents 90% of the total collagen of hyaline cartilage, and Type X is mainly associated with hypertrophic cartilage.

The specimens are dehydrated, embedded in paraffin and 5 μm sections are cut. For histochemical staining of GAG's 0.2% Toluidine blue are used. For the immunohistochemical analysis, the specimens are rehydrated, incubated in 1 mg/ml pronase (Sigma P-5147) in PBS (10 mM sodium phosphate, 0.15 M sodium chloride, pH 7.4) for 15 minutes at room temperature, and stained with antibodies to Type I, II and X collagen. Secondary antibody is biotinylated goat anti-mouse, diluted 1:100 in 1% BSA (bovine serum albumin) in PBS. The samples are then incubated in streptavidin-peroxidase, diluted 1:300 in 1% BSA/PBS. Slides are contrasted with a solution from Vector VIP Substrate Kit (Vectro Labs; Burlingame, Calif.) and counterstained with Fast Green. The slides are observed on a brightfield microscope.

Biochemistry

The samples are digested with 200 μg/ml papain in 20 mM sodium phosphate buffer, pH 6.8, 1 mM EDTA, 2 mM dithiothreitol for 180 minutes at 65° C. The samples are then incubated in 400 μl 0.1 N sodium hydroxide for 30 min with vortexing and neutralized with 400 μl of Neutralizing Buffer (5 M sodium chloride, 100 mM disodium phosphate, pH 7.2 plus 1/10 volume of 1.0 N hydrochloric acid). The sample is then mixed with an equal volume of 0.7 μg/ml of bis-benzimide (Hoechst dye #33258) in water, and the emission (455-465 nm) read with a VerseFluor fluorimeter (Bio-Rad) at an excitation wavelength of 340-380 nm. The total glycosaminoglycan content is assayed colorimetrically by a Safranin-O assay as previously described. All standard curves are fit by linear regression through the linear portion of the data. The sizes of sample aliquots are adjusted so that the absorbance values were within the linear portion of the standard curves. The final results are expressed as micrograms of glycosaminoglycan per mg wet weight and per μg of DNA.

dGEMRIC

Samples to be used for dGEMRIC staining are stored at −20° C. until ready. Samples are cut to 5×5 mm sizes to fit within the 9.4T Bruker MRI scanner, and up to six samples are placed in a customized non-magnetic holder. The samples are then incubated in 1 mM GdDTPA (Magnevist™, Berlex Inc.) in Hanks Balanced Salt Solution at 37° C. for 3 hours. Sample holders are then wrapped in an enclosed bag of saline to minimize air-holder interfaces and reduce problems with magnetic susceptibilities, and the wrapped samples are placed in the MR magnet at 37° C. A series of T1-weighted MR images are acquired with 500 μm-thick slices oriented perpendicular to the cartilage surface and with 125×125 micron in-plan resolution. Samples are then returned for continued growth studies or other analyses. Each MR image is analyzed by evaluating Gd-DTPA concentrations within the cartilage and in the bath using standard T1 relaxation analysis, and producing a spatial map of GAG content using the Donnan theory of Field Charge Distributions.

### EXAMPLE 3

### Introduction

The main goal of this research proposal is to develop functional tissue for use in tracheal reconstruction. These results seek to develop the methodology to produce tracheal tissue that has the necessary mechanical strength, shape, and is sufficiently vascularized to maintain a stable living tissue.

A total of 12 male New Zealand White rabbits, weighing between 3.0 kg and 3.5 kg, are used. The method for implantation is essentially the same as that described in the preliminary results, except that engineered cartilage is used in the construct instead of ear cartilage. An important change in the methodology that is different from the native tissue controls is that the engineered cartilage sheet is wrapped around the construct in multiple layers and secured with fibrin sealant (Tisseal®, Immuno AG, Vienna, Austria) and 5-0 vicryl sutures. Fibrin glue is applied between each layer in prevent shear movement and to allow them to integrate into each other. In addition, we are expecting this layering to promote annealing of the layers and an increase in mechanical strength. As in the preliminary results, the construct is then buried in a fascial pocket under the skin, followed by the wound closure. The samples are harvested at 4, 6, and 8 weeks post-implantation (4 samples per time point) and then assessed visually by histology and mechanical testing. The visual and histologic assessment is relatively straightforward as we are primarily interested in assessing vascularization, cartilage survival, the status of the cartilage sheets and whether they are annealing.

### Biomechanical Properties of a Native Trachea

The native cervical trachea of a rabbit is tested biomechanically for comparison to the engineered trachea. Tensile and bending configurations have been commonly used to determine mechanical characteristics of trachea. However, we believe that compressive behaviour is the critical mechanical characteristic of a clinically viable tracheal repair; that is, the repair must not collapse and obstruct the airway. Two approaches are used to evaluate compressive characteristics of the trachea.

In the first and simpler evaluation, a 1 cm length of trachea (native or engineered) is compressed between two smooth, flat platens, and the lumen photographed at prescribed values at applied force. Maximum applied force is that which reduces the lumen area of native trachea by 30%, a symptomatic level of stenosis. Measurements are made using a TestResources material testing machine, and lumen area is measured from digital images using ImageJ. Prior to testing, loose connective tissue is removed. A saline spray is used to maintain hydration. Results of this test are represented as change in lumen area as a function of force (a measure of area stiffness), and force at 30% area reduction.

In the second test a uniform pressure is applied to the exterior of the tracheal repair while lumen area is measured. Each end of a 2 cm section of trachea (native or repaired) is slid over stainless steel tubes, and secured with cyanoacrylate cement and suture wrapped around the trachea. Pressure is applied by varying the height of fluid in a tube attached to the test chamber. Lumen area is measured as described above.

From the above description of the invention, those skilled in the art will perceive improvements, changes and modifications. For example, it will be appreciated that the present invention may be used to repair other cartilage tissue defects, such as full- or partial-thickness cartilage defects of the knee, as well as any other cartilage tissue defects generally caused by osteoarthritis or other degenerative conditions. Such improvements, changes and modifications within the skill of the art are intended to be covered by the appended claims.

1-12. (canceled)

13. A hyaline-like, multilayer cartilage tissue construct comprising a plurality of layers having a glycosaminoglycan content substantially equal to the glycosaminoglycan content of native cartilage tissue, each of the layers comprising chondrogenic cells dispersed within an endogenously produced extracellular matrix.

14. The multilayer cartilage tissue construct of claim 13, the chondrogenic cells being autogenic, allogenic, or a combination thereof.

15. The multilayer cartilage tissue construct of claim 13, the chondrogenic cells being immature chondrocytes, mature chondrocytes, or a combination thereof.

16. The multilayer cartilage tissue construct of claim 13, the multilayer cartilage tissue construct having a thickness of about 1 mm to about 4 mm.

17. A method for generating a multilayer cartilage tissue construct, the method comprising the steps of:
isolating a population of chondrogenic cells;
expanding the population of chondrogenic cells;
seeding the population of chondrogenic cells into a bioreactor having a volume defined by oppositely disposed gas permeable membranes, the bioreactor including a serum-free culture medium;
culturing the population of chondrogenic cells in the bioreactor for a time sufficient to permit the population of chondrogenic cells to differentiate and form a single layer cartilage tissue construct;
removing the single layer cartilage tissue construct from the bioreactor; and
forming the single layer cartilage tissue construct into the multilayer cartilage tissue construct.

18. The method of claim 17, the step of forming the single layer cartilage tissue construct into the multilayer cartilage construct further comprising folding a first portion of the single layer cartilage tissue construct onto a second portion of the single layer cartilage tissue construct and then applying hydrostatic pressure, mechanical pressure, or a combination thereof to form the multilayer cartilage tissue construct.

19. The method of claim 17, the step of forming the single layer cartilage tissue construct into the multilayer cartilage construct further comprising overlaying at least one single layer cartilage tissue construct onto at least one other single layer cartilage tissue construct and then applying hydrostatic pressure, mechanical pressure, or a combination thereof to form the multilayer cartilage tissue construct.

20. The method of claim 17, the population of chondrogenic cells comprising immature chondrocytes, mature chondrocytes, or a combination thereof.

21. The method of claim 17, the population of chondrogenic cells being autogenic, allogenic, or a combination thereof.

22. The method of claim 17, the step of expanding the population of chondrogenic cells further comprising culturing the population of chondrogenic cells in a conditioned growth media effective to promote expansion of the population of chondrogenic cells.

23. The method of claim 17, the serum-free culture medium including at least one growth factor selected from the group consisting of transforming growth factor-β, platelet-derived growth factor, insulin-like growth factor, acid fibroblast growth factor, basic fibroblast growth factor, epidermal growth factor, hepatocytic growth factor, keratinocyte growth factor, and bone morphogenic protein.

24. The method of claim 17, the population of chondrogenic cells endogenously producing an extracellular matrix when the population of chondrogenic cells is cultured in the bioreactor.

25. The method of claim 17, the step of culturing the population of chondrogenic cells in the bioreactor further comprising growing the population of chondrogenic cells at about 37° C. in a humidified atmosphere with the addition of about 5% carbon dioxide and about 1% to about 21% oxygen.

26. A multilayer cartilage tissue construct produced by a method of claim 17.

27. A method for generating a tracheal implant, the method comprising the steps of:
isolating a population of chondrogenic cells;
expanding the population of chondrogenic cells;
seeding the population of chondrogenic cells into a bioreactor having a volume defined by oppositely disposed gas permeable membranes, the bioreactor including a serum-free culture medium;
culturing the population of chondrogenic cells in the bioreactor for a time sufficient to permit the population of chondrogenic cells to differentiate and form a single layer cartilage tissue construct;
removing the single layer cartilage tissue construct from the bioreactor; and
forming the tracheal implant with the single layer cartilage tissue construct.

28. The method of claim 27, the tracheal implant comprising a whole trachea.

29. The method of claim 28, the tracheal implant comprising a portion of the whole trachea.

30-33. (canceled)
